(19) United States
(12) Patent Application Publication
(10) Pub. No.: US 2026/0185040 A1
(43) Pub. Date: Jul. 2, 2026

(54) MARINE BACTERIUM FOR PRODUCING CAROTENOIDS AND ISOLATED CULTURE METHOD THEREOF

(71) Applicant: Ningbo University, Ningbo (CN)

(72) Inventors: Jiajie Xu, Ningbo (CN); Wenwen Zhang, Ningbo (CN); Wen Zhang, Ningbo (CN); Fei Kong, Ningbo (CN); Xiurong Su, Ningbo (CN)

(21) Appl. No.: 19/308,361

(22) Filed: Aug. 25, 2025

(30) Foreign Application Priority Data
Jan. 2, 2025 (CN) 202510000932.4

(51) Int. Cl. C12N 1/205; C12P 23/00; C12R 1/01

(52) U.S. Cl. CPC C12N 1/205; C12P 23/00; C12R 2001/01

(57) ABSTRACT

The present invention relates to the field of marine bacteria, and discloses a marine bacterium for producing carotenoids and an isolated culture method thereof. This strain is isolated from seawater, is named as Rossellomorea marisflavi NDS (R. marissflavii NDS), is preserved in the China General Microbiological Culture Collection Center (CGMCC), and has the preservation number of CGMCC NO. 32287 and the preservation date of Oct. 21, 2024. This strain is extracted from seawater, and has good eco-friendliness and oligotrophic characteristics. The raw materials of a medium are low in cost and easy to obtain. Meanwhile, this strain has an excellent growth speed and can reach the logarithmic phase within several hours, and complete the accumulation of carotenoids within 20 h. The produced carotenoids account for 1.764% of the dry cell weight, and the production efficiency is high.

### CROSS-REFERENCE TO RELATED APPLICATIONS

The Sequence Listing XML file entitled “Sequence Listing XML,” created on Dec. 11, 2024, and having a size of 3,334 bytes, is incorporated herein by reference in its entirety pursuant to 37 C.F.R. §§ 1.831-1.835.

### TECHNICAL FIELD

The present invention relates to the field of marine bacteria, in particular to a marine bacterium for producing carotenoids and an isolated culture method thereof.

### BACKGROUND ART

Carotenoids are a class of biologically active secondary metabolites with antioxidant, anti-inflammatory, and immune-enhancing effects. Studies have shown that providing carotenoid-containing feeds during a reproduction phase of fish can significantly improve the quality of eggs and increase the survival rate of juvenile fish. In addition, for colorful aquatic products such as rainbow trout and shrimps, carotenoids also play a vital role in maintaining the body color and the muscle health. However, natural carotenoids are mainly found in photosynthetic plants, bacteria, and microalgae. Natural sources of carotenoids are limited for most marine organisms, and concentrations are often insufficient to meet the needs of growth and reproduction, so appropriate amounts of carotenoids need to be supplemented in aquaculture feeds.

In industrial production, carotenoids are produced in two main ways: extraction from natural sources such as plants, algae, and microorganisms; and chemical synthesis. The most typical carotenoids that can be extracted from plants include: β-carotenoids extracted from carrots and pumpkins; lycopene extracted from tomatoes; zeaxanthin extracted from corn and red peppers, etc. There are varieties of algae such as Haematococcus pluvialis, Dunaliella salina, and chlorella. Under the conditions of high light, nitrogen deficiency or high salt stress, the yield of astaxanthin in each liter of Haematococcus pluvialis medium may reach 1.5-3% (in dry weight). In a large-scale culture system, the yield of β-carotene in each liter of Dunaliella salina medium may reach 3-5% (in dry weight). In commercial culture, the yield of carotenoids (a total amount of lutein and β-carotene) in each liter of chlorella medium is about 0.5-2% (in dry weight). The main bacteria that produce carotenoids include photosynthetic bacteria: purple sulfur bacteria, green sulfur bacteria, and Rhodospirillum; and non-photosynthetic bacteria include Flavobacterium, Pseudomonas, and Lactobacillus delbrueckii. However, the yields of carotenoids in these bacteria are relatively low, and the content of carotenoids in each liter of medium is only 0.1-1% (in dry weight). In addition, the types of bacteria that are easy for mass production are also relatively limited.

However, compared to plants and algae, bacteria have the advantages of faster reproduction speed and lower culture cost. Therefore, finding a bacterium that can efficiently produce carotenoids and is easy for mass production is crucial in terms of saving the culture time and cost. Meanwhile, since this bacterium eventually needs to be added to an aquaculture feed to increase the carotenoid content, it is also necessary to ensure that this bacterium, when added to seawater along with the aquaculture feed, has less influence on the marine ecology.

### SUMMARY OF THE INVENTION

A first object of the present invention is to provide a marine bacterium that can efficiently produce carotenoids, is easy for mass production, and has less influences on marine ecology after being added to seawater, in view of the shortcomings that bacteria that can produce carotenoids in the prior art are relatively low in yield and difficult for mass production.

In order to solve the above technical problems, the present invention adopts the following technical solutions.

A marine bacterium for producing carotenoids is provided, wherein this strain is named as Rossellomorea marisflavi NDS (R. marissflavii NDS), is preserved in the China General Microbiological Culture Collection Center (CGMCC), and has the preservation number of CGMCC NO. 32287 and the preservation date of Oct. 21, 2024.

By using the above solution, this bacterium is isolated from seawater obtained at a depth of 0.5 m in the central sea area (121°43′06″E, 29°34′44″N) of Xiangshan Port in Ningbo, Zhejiang Province. Through the analysis for a 16S rRNA gene sequence, it is found that this bacterium belongs to Rossellomorea, which has the highest homology with a strain Rossellomorea marisflavi A, both of which belong to Rossellomorea marisflavi. Therefore, this bacterium is named as Rossellomorea marisflavi NDS (R. marisflavi NDS). This strain is preserved in the China General Microbiological Culture Collection Center (CGMCC), and has the preservation number of CGMCC NO. 32287 and the preservation date of Oct. 21, 2024.

This strain is extracted from seawater, and has an excellent growth rate. The produced carotenoids account for 1.764% of the dry cell weight, and meet the standards for endotoxicity. Therefore, this bacterium can efficiently produce the carotenoids, is easy for mass production, can be symbiotic with marine organisms, and has eco-friendliness.

Preferably, the accumulation of the carotenoids is maximized when this bacterium grows to 20 h.

A second object of the present invention is to provide an isolated culture method for isolating the above-mentioned marine bacterium from seawater, and purifying and culturing the marine bacterium.

An isolated culture method of a marine bacterium is provided. The isolated culture method includes:

A1. isolation of the marine bacterium from seawater:
collecting a seawater sample, diluting and inoculating the seawater sample in a liquid medium, and culturing for 12-16 h at a temperature of 36-38° C. and a shaker speed of 100-150 r/min to form a bacterium culture solution;
coating the bacterium culture solution on a plate medium, picking mixed bacterium colonies after 10-14 h of culture, and performing streak culture on the plate medium to obtain a mixed bacterium culture;
diluting the mixed bacterium culture with sterile water to form a bacterium solution, pipetting the bacterium solution and evenly coating the bacterium solution on the liquid medium, culturing for 10-12 h at a temperature of 36-38° C. and a shaker speed of 100-150 r/min, then picking yellow colonies separately, and performing partitioned streak-purifying culture on the plate medium to form a purified bacterium; and
A2. culture of the marine bacterium:
inoculating the purified bacterium into a liquid medium with an initial pH of 7-9 at an inoculation amount of 0.1-0.15%, and culturing for 8-16 h at a temperature of 36-38° C. and a shaker speed of 100-150 r/min.

By using the above solution, the seawater sample is inoculated in the liquid medium to form mixed bacterium colonies; the mixed bacterium colonies are subjected to streak culture on the plate medium to obtain the mixed bacterium culture; the mixed bacterium culture is coated on the liquid medium; yellow colonies are picked separately after the culture, and then subjected to streak-purifying culture on the plate medium to form the purified bacterium; and the purified bacterium is then stored for subsequent experiments, wherein the purified bacterium is R. marisflavi NDS.

In subsequent experiments, the purified R. marisflavi NDS is cultured first, and the growth of R. marisflavi NDS is observed, followed by endotoxin content determination and carotenoid content determination for R. marisflavi NDS.

The results showed that R. marisflavi NDS can be symbiotic with marine organisms, and has eco-friendliness because R. marisflavi NDS contains endotoxin below a specified limit and can be isolated and extracted from seawater. Meanwhile, R. marisflavi NDS has an excellent growth speed. In addition, by comparing the growth and intracellular pigment accumulation of R. marisflavi NDS, it is found that the accumulation of pigments in R. marisflavi NDS is maximized when R. marisflavi NDS grows to a plateau phase of 20 h, and it is measured by sampling at corresponding time points that the carotenoids in R. marisflavi NDS account for 1.764% of the dry cell weight, which is significantly superior to that of other bacteria.

Preferably, the liquid medium is a liquid beef paste peptone medium, which contains the following components: 0.3% of beef paste, 1% of peptone, 2% of sodium chloride, and the balance of water.

Preferably, the plate medium is a solid beef paste peptone medium, which contains the following components: 0.3% of beef paste, 1% of peptone, 2% of sodium chloride, 2% of agar, and the balance of water.

By using the above solutions, the isolated R. marisflavi NDS has oligotrophic characteristics; and the used medium is the beef paste peptone medium, without additional carbon or nitrogen sources, so the raw materials are low in cost and easy to obtain.

By using the above technical solutions, the present invention has significant technical effects: this R. marisflavi NDS is isolated from seawater and cultured in the beef paste peptone medium with an excellent growth speed, and can reach the logarithmic phase within a few hours, and complete rapid accumulation of carotenoids in the plateau phase. The produced carotenoids account for 1.764% of the dry cell weight and meet the standards for endotoxicity. Therefore, this bacterium can efficiently produce the carotenoids, is easy for mass production, can be symbiotic with marine organisms, and has eco-friendliness.

### BRIEF DESCRIPTION OF THE DRAWINGS

FIG. 1 is a schematic diagram of R. marisflavi NDS on a plate medium in an example;

FIG. 2 is a transmission electron microscope photograph of R. marisflavi NDS in an example;

FIG. 3 is a 16s rRNA phylogenetic tree of R. marisflavi NDS in an example;

FIG. 4 is a growth curve diagram of R. marisflavi NDS in an example; and

FIG. 5 is a comparison diagram of the growth and pigment accumulation of R. marisflavi NDS in an example.

### DETAILED DESCRIPTION OF THE INVENTION

The present invention will be further described below in conjunction with the accompanying drawings and examples.

### EXAMPLES

A marine bacterium for producing carotenoids and an isolated culture method thereof are disclosed, which include a marine bacterium and a method for separating the marine bacterium from seawater, and purifying and culturing the marine bacterium.

1. Isolation of bacterium:
a seawater sample is collected at a depth of 0.5 m in the central sea area (121°43′06″E, 29° 34′44″N) of Xiangshan Port in Ningbo, Zhejiang Province.

50 L of seawater sample was taken, diluted by a factor of 8-12 and then inoculated in a liquid medium, and cultured for 12-16 h at a temperature of 36-38° C. and a shaker speed of 100-150 r/min to form a bacterium culture solution. In the present example, the seawater sample was diluted by a factor of 10, and cultured for 14 h at a temperature of 37° C. and a shaker speed of 120 r/min; and the medium was a liquid beef paste peptone medium. The liquid beef paste peptone medium contained the following components: 0.3% of beef paste, 1% of peptone, 2% of sodium chloride, and the balance of water.

100 L of bacterium culture solution was coated on a plate medium; and after 10-14 h of culture, mixed bacterium colonies with good growth and different morphologies were picked for streak culture to obtain a mixed bacterium culture. In the present example, the culture duration was 12 h, and the plate medium was a solid beef paste peptone medium. The solid beef paste peptone medium contained the following components: 0.3% of beef paste, 1% of peptone, 2% of sodium chloride, 2% of agar, and the balance of water.

The mixed bacterium culture was adsorbed using glycerol magnetic beads to form a bacterium glycerol preservation solution, which was then stored in an −80° C. refrigerator. The glycerol magnetic beads and the adsorption method of the glycerol magnetic beads for the mixed bacterium culture were both of the prior art and will not be repeated here. In addition, the glycerol magnetic beads only had an effect of prolonging the storage duration of the mixed bacterium culture, and did not affect subsequent purification and culture and subsequent experiments of the bacterium.

10 L of bacterium glycerol preservation solution was taken, and diluted with sterile water by a factor of 10-20 to form a bacterium solution. In the present example, the bacterium glycerol preservation solution was diluted with sterile water by a factor of 12.

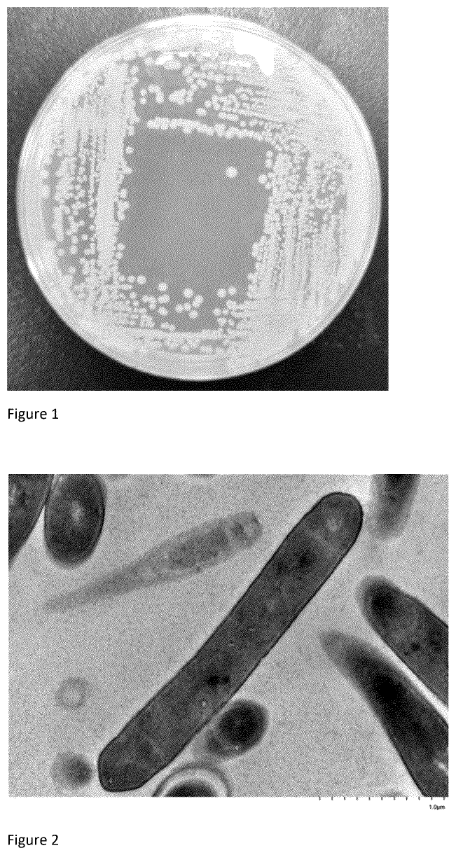

100 L of bacterium solution was pipetted and evenly coated on the liquid medium, and cultured for 10-12 h at a temperature of 36-38° C. and a shaker speed of 100-150 r/min; yellow colonies were then picked separately, followed by partitioned streak-purifying culture on the plate medium to form a purified bacterium, see FIG. 1 for details. In the present example, the temperature was 37° C., the shaker speed was 120 r/min, and the culture duration was 11 h.

The purified bacterium was stored in an −80° C. refrigerator.

2. Culture of bacterium:
the purified bacterium was inoculated into a liquid medium with an initial pH of 7-9 at an inoculation amount of 0.1-0.15%, and cultured at a temperature of 36-38° C. and a shaker speed of 100-150 r/min for 8-16 h. In the present example, the initial pH of the liquid medium was 7.5, the inoculation amount was 0.12%, the culture temperature was 37° C., and the shaker speed was 120 r/min.

When the bacterium was activated to the logarithmic phase, the OD₆₀₀ value reached 0.4-0.8. The purified strain was placed under an transmission electron microscope to observe the morphologies of the bacterium, see FIG. 2 for details. The transmission electron microscope photograph showed that a bacterium cell was surrounded by a dense shell, and the internal contents were separated by an inner membrane. The cell was in a shape of a long pole, with a length of 3.07 m and a width of 431 nm.

3. Identification and naming of bacterium

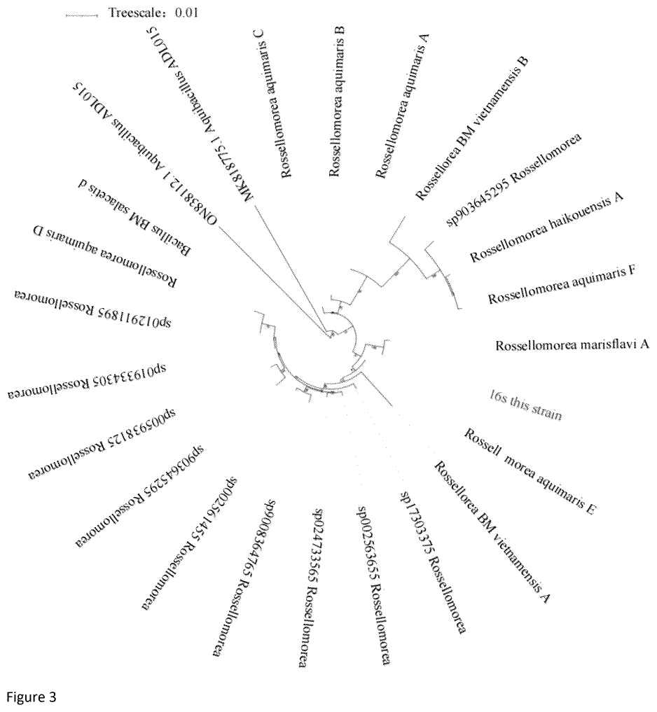

A phylogenetic location of this bacterium was determined by analyzing a 16S rRNA gene sequence. DNA of this strain was isolated, and a 16S rRNA gene was amplified by PCR using universal bacterium primers 27F and 1492R. The 16S rRNA sequence of this bacterium was aligned with a GeneBank online database using a BLASTN program, and a phylogenetic tree was constructed using Mega 7.0 software, see FIG. 3 for details.

16s rRNA sequence of Rossellomorea marisflavi:

GTTTGATCCTGGCTCAGGACGAACGCTGGCGGCGTGCCTAATACATGC
AAGTCGAGCGGATCGATGGGAGCTTGCTCCCTGAGATCAGCGGCGGACG
GGTGAGTAACACGTGGGTAACCTGCCTGTAAGACTGGGATAACTCCGGG
AAACCGGGGCTAATACCGGATAACACCTACCCCCGCATGGGGGAAGGTT
GAAAGGTGGCTTCGGCTATCACTTACAGATGGACCCGCGGCGCATTAGC
TAGTTGGTGAGGTAACGGCTCACCAAGGCGACGATGCGTAGCCGACCTG
AGAGGGTGATCGGCCACACTGGGACTGAGACACGGCCCAGACTCCTACG
GGAGGCAGCAGTAGGGAATCTTCCGCAATGGACGAAAGTCTGACGGAGC
AACGCCGCGTGAGTGAAGAAGGTTTTCGGATCGTAAAACTCTGTTGTTA
GGGAAGAACAAGTGCCGTTCGAATAGGGCGGCACCTTGACGGTACCTAA
CCAGAAAGCCACGGCTAACTACGTGCCAGCAGCCGCGGTAATACGTAGG
TGGCAAGCGTTGTCCGGAATTATTGGGCGTAAAGCGCGCGCAGGTGGTT
TCTTAAGTCTGATGTGAAAGCCCACGGCTCAACCGTGGAGGGTCATTGG
AAACTGGGGAACTTGAGTGCAGAAGAGGAAAGTGGAATTCCAAGTGTAG
CGGTGAAATGCGTAGATATTTGGAGGAACACCAGTGGCGAAGGCGACTT
TCTGGTCTGTAACTGACACTGAGGCGCGAAAGCGTGGGGAGCAAACAGG
ATTAGATACCCTGGTAGTCCACGCCGTAAACGATGAGTGCTAAGTGTTA
GAGGGTTTCCGCCCTTTAGTGCTGCAGCTAACGCATTAAGCACTCCGCC
TGGGGAGTACGGTCGCAAGACTGAAACTCAAAGGAATTGACGGGGGCCC
GCACAAGCGGTGGAGCATGTGGTTTAATTCGAAGCAACGCGAAGAACCT
TACCAGGTCTTGACATCCTCTGACAACCCTAGAGATAGGGCTTTCCCCT
TCGGGGGACAGAGTGACAGGTGGTGCATGGTTGTCGTCAGCTCGTGTCG
TGAGATGTTGGGTTAAGTCCCGCAACGAGCGCAACCCTTGATCTTAGTT
GCCAGCATTCAGTTGGGCACTCTAAGATGACTGCCGGTGACAAACCGGA
GGAAGGTGGGGATGACGTCAAATCATCATGCCCCTTATGACCTGGGCTA
CACACGTGCTACAATGGACGGTACAAAGGGCTGCAAGACCGCGAGGTTT
AGCCAATCCCATAAAACCGTTCTCAGTTCGGATTGTAGGCTGCAACTCG
CCTACATGAAGCTGGAATCGCTAGTAATCGCGGATCAGCATGCCGCGGT
GAATACGTTCCCGGGCCTTGTACACACCGCCCGTCACACCACGAGAGTT
TGTAACACCCGAAGTCGGTGAGGTAACCTTTTGGAGCCAGCCGCCTAAG
GTGGGACAGATGATTGGGGTGAAGTCGTAACAAGGTAGCCGTA.

Through the analysis of morphological characteristics, 16S rDNA and whole genome sequencing, it was identified that this strain belonged to Rossellomorea, which had the highest homology with a strain Rossellomorea marisflavi A, and was named as R. marisflavi NDS.

4. Determination of the growth curve of bacterium

A liquid medium was prepared and sterilized at 121° C. for 20 min. The liquid medium was aliquoted into 50 mL cell culture flasks, with 25 mL in each flask. Individual colonies were picked and cultured under shaking for 10 h at a temperature of 37° C. and a shaking speed of 120 r/min, and the OD₆₀₀ value (i.e., a primary bacterium solution) was measured. Then, 10 L of primary bacterium solution was transferred to a culture flask and continued to culture (i.e., a secondary bacterium solution) under the same conditions, and the OD₆₀₀ value was measured every 4 h. The results were shown in FIG. 4. It was found that the concentration of R. marisflavi NDS began to increase significantly after 6 h of culture. During the period of 6-14 h, the bacterium grew rapidly and proliferated in large quantities, entering the logarithmic phase. After 14 h of culture, the concentration of the bacterium solution did not change much, indicating that the cells had grown to the plateau phase.

5. Endotoxicity determination of bacterium

Bacterium endotoxins were a group of lipopolysaccharide (LPS) complexes that existed widely inside and outside cells and could produce certain toxic (sensitized/pathogenic) effects on normal physiological activities of surrounding organisms during the growth and reproduction process of bacteria. In order to determine whether the endotoxin content of the isolated strain was within a permissible range, an endotoxin detection reagent (TAL chromogenic method) was used to quantitatively detect the endotoxin content in this bacterium. The calculation formula was as follows: L=MVD×λ/c, wherein L represented a bacterium endotoxin limit of a test sample, MVD was a maximum effective dilution factor, and λ was the minimum endotoxin concentration in a standard curve. The method for determining the bacterium endotoxin was of the prior art and would not be repeated here.

It was calculated in an experiment that the endotoxin content in R. marisflavi NDS was 0.085 EU/mL, which was 0.1 EU/mL below a specified limit (Chinese Pharmacopoeia). Therefore, it could be determined that the endotoxin content in R. marisflavi NDS was in a low level, which meant that this strain was less likely to cause diseases and sensitization to other organisms during growth, and had high biosecurity.

6. Determination of carotenoid production content in bacterium

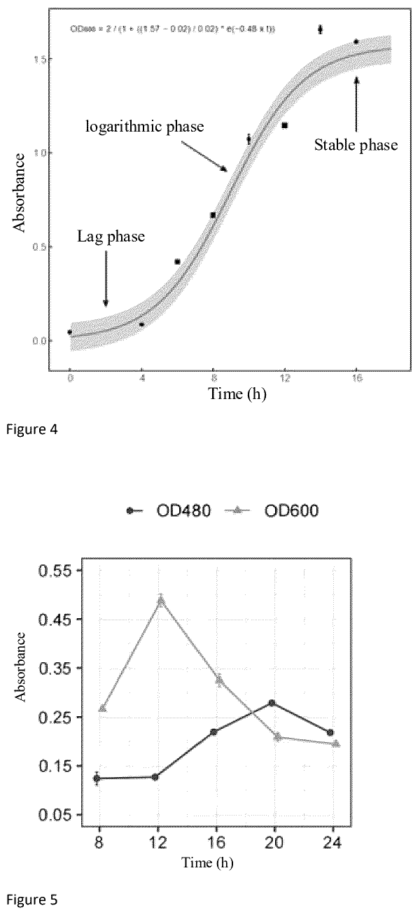

By comparing a relationship between the growth (OD₆₀₀) of the bacterium and pigment accumulation in cells (OD₄₈₀), it was found that the pigment accumulation was maximized when R. marisflavi NDS grew to 20 h. The results were shown in FIG. 5. In view of this, bacterium cells which were cultured for 20 h were collected, and a yellow pigment in the bacterium cells was extracted using a mixed organic solvent. The mixed organic solvent included methanol and trichloromethane at a ratio of 1:2.

The bacterium cells were added to the mixed organic solvent, disrupted by ultrasonication for 25 min at 200-250W at room temperature in dark place, and then centrifuged at 8000×g for 10 min. Several extractions were repeated until a cell debris presented white and were free of pigments.

A supernatant was collected, and the absorbance at OD₄₈₀ was tested. The pellet was resuspended in 1× PBS buffer to obtain a resuspension solution. The resuspension solution was dried to a constant weight in an oven at 60° C.

The total carotenoid content (TCC, g/g) was determined using the following formula: TCC=A×D×V/(E×W), wherein A was the absorbance of totally extracted carotenoids at 480 nm; D was a sample dilution ratio; V was a volume (mL) of an extraction solvent; E was an extinction coefficient (0.16) of carotenoids; and W was a dry weight (g) of bacterium cells.

R. marisflavi NDS could accumulate 1.764 g/100 g of carotenoids just by 20 h, accounting for 1.764% of the dry cell weight. The production efficiency was high.

In summary, R. marisflavi NDS was extracted from seawater and met the standards for endotoxin content. Therefore, this bacterium was added to the ocean as a carotenoid supplement along with a production feed, and thus was friendly to the marine ecology. R. marisflavi NDS could be cultured with the beef paste peptone medium, without additional carbon or nitrogen sources, so the raw materials are low in cost and easy to obtain. Meanwhile, R. marisflavi NDS had an excellent growth speed, could reach a logarithmic phase within a few hours, and was simple and fast to culture, and easy to operate. 1.764 g/100 g of carotenoids could be accumulated just by 20 h, accounting for 1.764% of the dry cell weight, so the production efficiency was high.

Therefore, R. marisflavi NDS was a marine bacterium that could efficiently produce carotenoids, was easy for mass production and had less influences on marine ecology.

The above is only preferred embodiments of the present invention, the protection scope of the present invention is not limited to the above examples, all technical solutions belonging to the ideas of the present invention are within the protection scope of the present invention. It should be noted that those of ordinary skill in the art may also make several improvements and modifications without departing from the principles of the present invention, which should be considered as the protection scope of the present invention.

1. A marine bacterium for producing carotenoids, wherein the strain is named as Rossellomorea marisflavi NDS, is preserved in the China General Microbiological Culture Collection Center (CGMCC), and has the preservation number of CGMCC NO. 32287 and the preservation date of Oct. 21, 2024.

2. The marine bacterium for producing carotenoids according to claim 1, wherein the accumulation of the carotenoids is maximized when the strain grows to 20 h.


SEQUENCE LISTING (1 sequence)

SEQ ID NO: 1 - RNA, 1512 nt, synthetic construct
  source 1..1512: mol_type = other RNA
gtttgatcctggctcaggacgaacgctggcggcgtgcctaatacatgcaagtcgagcggatcgatgggagcttgctccctgagatcagcggcggacgggtgagtaacacgtgggtaacctgcctgtaagactgggataactccgggaaaccggggctaataccggataacacctacccccgcatgggggaaggttgaaaggtggcttcggctatcacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgaagaaggttttcggatcgtaaaactctgttgttagggaagaacaagtgccgttcgaatagggcggcaccttgacggtacctaaccagaaagccacggctaactacgtgccagcagccgcggtaatacgtaggtggcaagcgttgtccggaattattgggcgtaaagcgcgcgcaggtggtttcttaagtctgatgtgaaagcccacggctcaaccgtggagggtcattggaaactggggaacttgagtgcagaagaggaaagtggaattccaagtgtagcggtgaaatgcgtagatatttggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggctttccccttcgggggacagagtgacaggtggtgcatggttgtcgtcagctcgtgtcgtgagatgttgggttaagtcccgcaacgagcgcaacccttgatcttagttgccagcattcagttgggcactctaagatgactgccggtgacaaaccggaggaaggtggggatgacgtcaaatcatcatgccccttatgacctgggctacacacgtgctacaatggacggtacaaagggctgcaagaccgcgaggtttagccaatcccataaaaccgttctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtgaggtaaccttttggagccagccgcctaaggtgggacagatgattggggtgaagtcgtaacaaggtagccgta